(19) United States
(12) Patent Application Publication
(10) Pub. No.: US 2020/0237350 A1
(43) Pub. Date: Jul. 30, 2020

(54) DIGESTIVE TRACT MULTI-REGION LIQUID BIOPSY SAMPLING DEVICE

(71) Applicant: Ankon Medical Technologies (Shanghai) Co., LTD., Shanghai (CN)

(72) Inventors: Xiaodong Duan, Pleasanton, CA (US); Lei LIU, Suzhou (CN)

(73) Assignee: Ankon Medical Technologies (Shanghai) Co., LTD., Shanghai (CN)

(21) Appl. No.: 16/775,762

(22) Filed: Jan. 29, 2020

(30) Foreign Application Priority Data
Jan. 29, 2019 (CN) 201910086587.5

(51) Int. Cl. A61B 10/00; A61B 5/055; A61B 6/12

(52) U.S. Cl. CPC A61B 10/0045; A61B 2010/0061; A61B 6/12; A61B 5/055

(57) ABSTRACT

The present invention provides a digestive tract multi-region liquid biopsy sampling device. The digestive tract multi-region liquid biopsy sampling device includes an enclosure, and a plurality of sampling chambers formed in the enclosure. The enclosure includes through holes cut in the wall of the enclosure corresponding to each sampling chamber, and targeted dissolution membranes covering the through holes on different sampling chambers that can dissolve at different targeted regions. Expandable materials that can absorb liquids, arranged in the sampling chambers at the positions of the expandable materials corresponding to the through holes, therefore the expandable materials expand after absorbing liquid and close the through holes.

### CROSS-REFERENCE OF RELATED APPLICATIONS

The application claims priority to Chinese Patent Application No. 201910086587.5 filed on Jan. 29, 2019, the contents of which are incorporated by reference herein.

### FIELD OF INVENTION

The present invention relates to a medical device, and more particularly to a digestive tract multi-region liquid biopsy sampling device.

### BACKGROUND

Gastrointestinal biopsy technique is of great significance for the diagnosis of gastrointestinal disorders. Traditional gastrointestinal biopsy technique refers to using tissue clamps, electrosurgical tools, and puncture needles to obtain liquid for biopsy or diseased tissues with the assistance of endoscopy. For example, under endoscopy, tissue clamps directly obtain diseased tissues, and endoscopic mucosal resection/endoscopic submucosal dissection (EMR/ESD) and endoscopic ultrasound fine needle aspiration (EUS-FNA), etc. This biopsy method brings great pain to the patient. It is demanding for the operating skills of physician and is accompanied by risks such as bleeding, artificial ulcers, tissue fibrosis, and perforation of mucosa.

With the development of micro-machining technology, micro-electromechanical technology (MEMS) represented by capsule endoscopy has opened up a new way for minimally invasive/comfortable biopsy sampling of digestive tract. However, these techniques still face problems such as high cost, complex energy supply equipment, and insufficient positioning accuracy in the process of gastrointestinal biopsy.

In addition, the existing gastrointestinal biopsy technology can only sample liquid at one region of the digestive tract at a time, which can prolong the diagnosis of disease and increase the pain of patient.

### SUMMARY OF THE INVENTION

The present invention provides a digestive tract multi-region liquid biopsy sampling device. The digestive tract multi-region liquid biopsy sampling device can accurately sample liquid at a plurality of targeted regions without an external power source, and can protect the sampled liquid from being contaminated by the liquid at other regions.

The present invention provides a digestive tract multi-region liquid biopsy sampling device, comprising:

an enclosure, wherein the enclosure comprises a plurality of sampling chambers formed in the enclosure, through holes cut in the wall of the enclosure corresponding to each sampling chamber, and targeted dissolution membranes covering the through holes on different sampling chambers that dissolve at different targeted regions; and
expandable materials that absorb liquids, arranged in the sampling chambers at the positions of the expandable materials corresponding to the through holes, wherein the expandable materials expand after absorbing liquid and close the through holes.

Further, the enclosure is in a shape of capsule.

Further, each sampling chamber comprises water absorbing materials that absorb liquid.

Further, the expandable materials are a polymer compounded by rubber-based polymer and water-absorbent resin, and the expandable materials expand after absorbing liquid to block the through holes, so that the through holes are closed.

Further, the expandable materials are a polymer compounded by rubber-based polymer and water-absorbent resin, wherein the enclosure further comprises piston rings arranged in the sampling chambers, and each piston ring comprises a flow passage for liquid to flow through the piston ring,

wherein each piston ring is configured with its outer wall against the inner wall of the enclosure, the expandable materials are disposed at a side of the piston ring away from the through holes, and after absorbing liquid, the expandable materials expand to push the piston ring to block the through holes.

Further, the expandable materials compounded by rubber-based polymer and water-absorbent resin are granular, and the granular expandable materials are disposed on the water absorbing materials or the inner wall of the enclosure.

Further, the expandable materials are water absorbing materials, the enclosure further comprises piston rings, and each piston ring comprises a flow passage for liquid to flow through the piston ring, wherein each piston ring is configured with its outer wall against the inner wall of the enclosure, the water absorbing materials are disposed at a side of the piston ring away from the through holes, and after absorbing liquid, the water absorbing materials expand to push the piston ring to block the through holes.

Further, before absorbing liquid, the water absorbing material is in a compressed state.

Further, the enclosure further comprises tracer particles are arranged in the enclosure to display the position of the digestive tract multi-region liquid biopsy sampling device in the digestive tract through medical imaging technique.

Further, the enclosure further comprises balance weights disposed inside.

Further, the targeted dissolution membrane is selected from EUDRAGIT E100, a mixture of EUDRAGIT L100-55 and EUDRAGIT Plastoid B, pectin, guar gum, or a mixture of EUDRAGIT S100 and EUDRAGIT Plastoid B.

In summary, the present invention provides an enclosure and a plurality of sampling chambers in the enclosure with through holes cut in the wall of the enclosure corresponding to each sampling chamber. Different targeted dissolution membranes that can dissolve at different targeted regions cover the through holes of each sampling chamber. In addition, expandable materials that can expand after absorbing liquid are arranged in the enclosure. Therefore, the liquid at a plurality of regions can be accurately sampled without external power source and the through holes can be blocked by the expandable materials to protect the sampled liquids from being contaminated by the liquid at other regions.

The above description is only an overview of the technical solutions of the invention. For a thorough understanding of the technical means of the invention, and implementation in accordance with the specification, and that the above-described and other objects, features and advantages of the invention can be more clearly understood, detailed description of the preferred embodiments can be described in detail with reference to the accompanying drawings.

### BRIEF DESCRIPTION OF THE DRAWINGS

FIG. 1 shows a structural view of a digestive tract multi-region liquid biopsy sampling device according to the first embodiment of the invention.

FIG. 2 shows a structural view of the digestive tract multi-region liquid biopsy sampling device of FIG. 1 after a first targeted dissolution membrane is dissolved.

FIG. 3 shows a structural view of the digestive tract multi-region liquid biopsy sampling device of FIG. 1 after a second targeted dissolution membrane is dissolved.

FIG. 4 shows a structural view of the digestive tract multi-region liquid biopsy sampling device of FIG. 1 after sampling is completed.

FIG. 5 shows a structural view of a digestive tract multi-region liquid biopsy sampling device according to the second embodiment of the invention.

FIG. 6 shows a structural view of a piston ring in FIG. 5.

FIG. 7 shows a structural view of the digestive tract multi-region liquid biopsy sampling device of FIG. 5 after sampling is completed.

FIG. 8 shows a structural view of the digestive tract multi-region liquid biopsy sampling device according to the third embodiment of the invention.

FIG. 9 shows a structural view of the Digestive tract multi-region liquid biopsy sampling device of FIG. 8 after sampling is completed.

### DETAILED DESCRIPTION

The present invention can be described in detail below with reference to the accompanying drawings and preferred embodiments.

The present invention provides a digestive tract multi-region liquid biopsy sampling device. The digestive tract multi-region liquid biopsy sampling device can accurately sample liquid at a plurality of targeted regions without an external power source, and can protect the sampled liquid from being contaminated by the liquid at other regions.

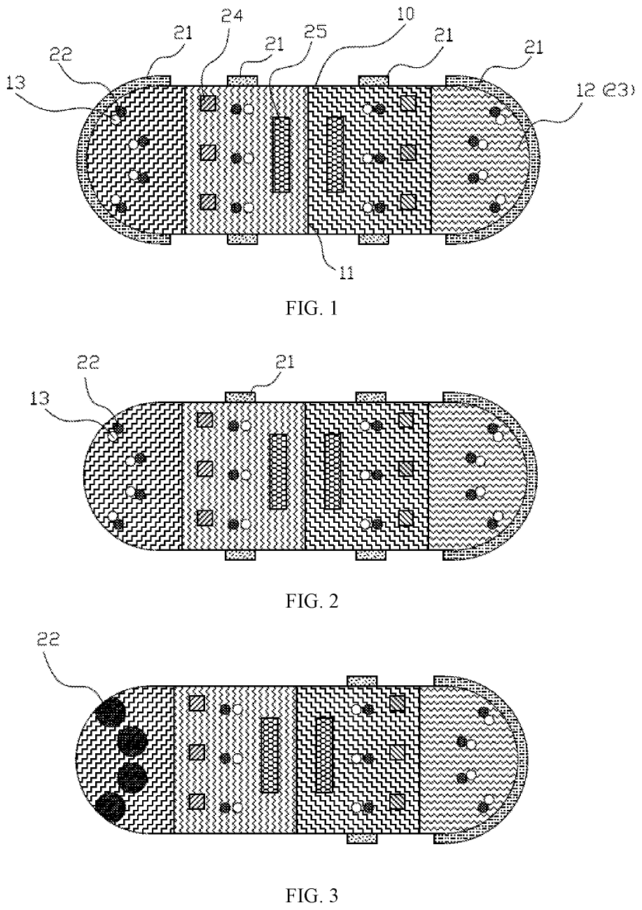

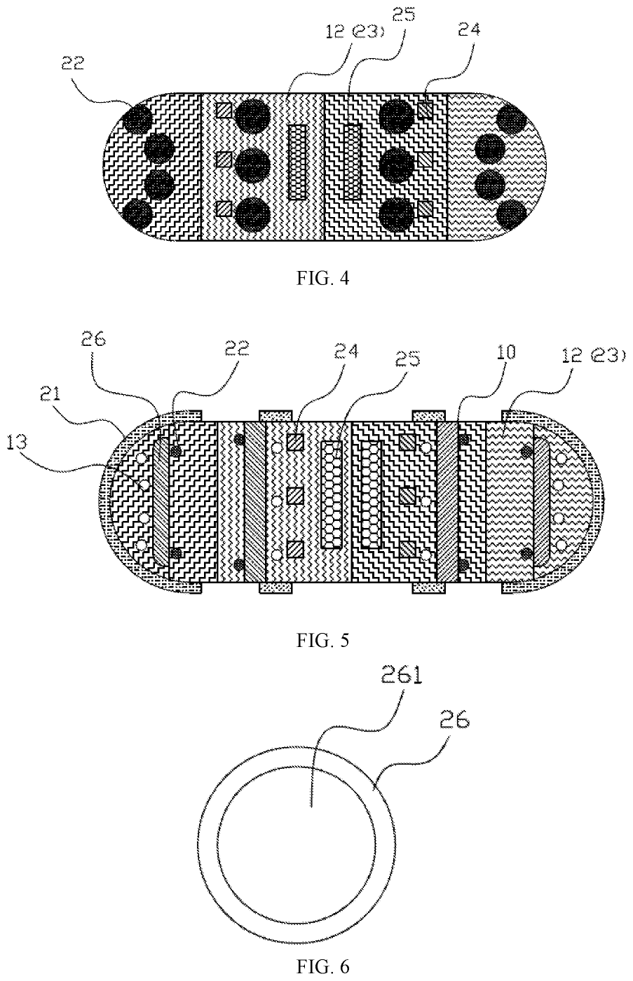

FIG. 1 shows a structural view of the digestive tract multi-region liquid biopsy sampling device according to the first embodiment of the invention. FIG. 2 shows a structural view of the digestive tract multi-region liquid biopsy sampling device of FIG. 1 after a first targeted dissolution membrane is dissolved. FIG. 3 shows a structural view of the digestive tract multi-region liquid biopsy sampling device of FIG. 1 after a second targeted dissolution membrane is dissolved. FIG. 4 shows a structural view of the digestive tract multi-region liquid biopsy sampling device of FIG. 1 after sampling is completed. Referring to FIGS. 1-4, the digestive tract multi-region liquid biopsy sampling device according to the first embodiment of the invention comprises an enclosure 10, a plurality of partitions 11 in the enclosure 10 to partition the enclosure 10 into a plurality of sampling chambers 12. The enclosure 10 comprise through holes 13 cut in the wall of the enclosure 10 corresponding to each sampling chamber 12, and targeted dissolution membranes 21 covering the through holes 13 of different sampling chambers 12 that can dissolve at different regions. The targeted dissolution membrane 21 on each sampling chamber 12 covers the through holes 13 on the sampling chamber 12. Expandable materials 22 that can absorb liquids to expand are arranged in each sampling chamber 12 at the positions corresponding to the through holes 13. The expandable materials 22 expand after absorbing liquid and close the through holes 13.

Referring to FIGS. 1-4, in the embodiment, four sampling chambers 12 can be partitioned, and the targeted regions of the four sampling chambers 12 can be the stomach, duodenum, jejunum, and colon. It can be understood that, in other embodiments, the number of the sampling chambers 12 and the corresponding targeted regions may have other options according to actual conditions, and is not limited thereto. For example, the number of the sampling chambers 12 can be two, three, or five.

In the embodiment, the present invention provides an enclosure 10 and a plurality of sampling chambers 12 in the enclosure 10 with through holes 13 cut in the wall of the enclosure 10 corresponding to each sampling chamber 12. Different targeted dissolution membranes 21 that can dissolve at different targeted regions cover the through holes 13 of each sampling chamber 12. In addition, expandable materials 22 that can expand after absorbing liquid are arranged in the enclosure 10. When the sampling device reaches the stomach, the targeted dissolution membrane 21 that can dissolve in the stomach dissolves, while other dissolution membranes do not change. At this time, liquid in stomach flows into the sampling chamber 12 corresponding to stomach through the through holes 13 on the sampling chamber 12 (see the left sampling chamber 12 in FIG. 2). As liquid enters, the expandable materials 22 in the sampling chamber 12 absorbs the liquid to expand, and then close the through holes 13, so that the liquid outside can no longer enter the sampling chamber 12. When the sampling device reaches the next targeted region, such as the duodenum, the targeted dissolution membrane 21 that can dissolve in the duodenum dissolves, while other dissolution membranes do not change. At this time, liquid in duodenum flows into the sampling chamber 12 corresponding to duodenum through the through holes 13 on the sampling chamber 12 (see the left second sampling chamber 12 in FIG. 2) and the expandable materials 22 in the sampling chamber 12 corresponding to duodenum absorb the liquid and expand to close the through holes 13. As the sampling device passes through the jejunum and colon successively, the liquid in the jejunum and colon enters the sampling device, so that sampling of a plurality of regions is completed. Therefore, the digestive tract multi-region liquid biopsy sampling device disclosed herein can accurately sample liquid at a plurality of targeted regions without an external power source, and can protect the sampled liquid from being contaminated by the liquid at other regions due to an arrangement of expandable materials 22 that can expand after absorbing liquid to close the through holes 13.

In the embodiment, the enclosure 10 can be made of materials such as medical grade polycarbonate, polyurethane, polyacrylate, polymethyl methacrylate, polyetheretherketone, polystyrene, or polyethylene. In the embodiment, in order to facilitate the movement of the sampling device in the digestive tract, the enclosure 10 can be in a shape of capsule.

In the embodiment, in order to improve sampling of liquid, each sampling chamber 12 further comprises a water absorbing material 23 inside. The water absorbing material 23 can be a water absorbing sponge, such as medical polyvinyl alcohol (PVA) sponge, polyurethane (PU) sponge, cotton fiber sponge, lignocellulose sponge, and/or chitosan sponge. In other embodiments, the water-absorbing material 23 can also be sodium polyacrylate-modified lignocellulose or chitosan-modified lignocellulose.

In the embodiment, the expandable materials 22 can be a polymer compounded by a rubber-based polymer and a water-absorbing resin, such as polyether-modified polyurethane rubber, sodium polyacrylate-modified styrene-butadiene rubber, polytetrahydrofuran-modified butadiene rubber, and acrylamide-modified ethylene-propylene-diene rubber and the like. The expandable materials 22 can be granular, and the granular expandable materials 22 are fixed on the water-absorbing material 23 or the inner wall of the enclosure 10, and the positions of the expandable materials 22 correspond to the through holes 13. After absorbing liquid, the expandable materials 22 expand to block the through holes 13, so that the through holes 13 are closed. The expansion rate of the expandable materials 22 can be determined according to the actual situation to ensure that sufficient liquid enters the sampling chamber 12 at a targeted region, and at the same time, ensure that the expandable materials 22 can close the through holes 13 before the sampling device leaves the targeted region.

In the embodiment, the targeted dissolution membranes 21 can cover the through holes 13 from the outside of the enclosure 10 or the inside of the enclosure 10 to prevent outside liquid from flowing into the sampling chamber 12 in a state that the membranes are not dissolved yet. The material of the targeted dissolution membranes 21 that dissolve at a targeted region can be determined according to the targeted region. For example, when stomach is the targeted region, the targeted dissolution membranes 21 dissolve in stomach, and the material of the targeted dissolution membranes 21 can be EUDRAGIT E100. When duodenum is the targeted region, the targeted dissolution membranes 21 can resist the erosion of acidic liquid in the stomach and dissolve in the duodenum due to high pH to release the drug, for example, the material of the targeted dissolution membranes 21 can be a mixture of EUDRAGIT L100-55 and EUDRAGIT Plastoid B. When colon is the targeted region, the targeted dissolution membranes 21 can dissolve due to the glycosidases and glycanase produced by special flora in colon to release the drug, for example, the material of the targeted dissolution membranes 21 can be pectin and/or guar gum. In another embodiment, when colon is the targeted region, the targeted dissolution membranes 21 can dissolve in the colon due to high pH to release the drug, for example, the material of the targeted dissolution membranes 21 can be a mixture of EUDRAGIT S100 and EUDRAGIT Plastoid B. In the embodiment, in order to ensure that the targeted dissolution membranes 21 of the gastrointestinal liquid biopsy sampling device can dissolve at the targeted region, the thickness of the targeted dissolution membranes 21 can be 100 nm-100 μm.

In one embodiment, the enclosure 10 further comprises tracer particles 24 inside. The tracer particles 24 can display the position of the sampling device in digestive tract under X-ray or magnetic field scanning The tracer particles 24 can be barium sulfate particles or magnetic metal particles, and the tracer particles 24 have anti-corrosion coatings such as parylene coating or Parylene-C coating membrane, to prevent the liquid in the digestive tract from corroding the tracker particles 24. In this embodiment, to facilitate the fixation of the tracker particles 24, the tracker particles 24 can be fixed in the water absorbing material 23.

In the embodiment, to enable the sampling device to be immersed in the liquid in digestive tract for easy sampling of liquid, the digestive tract multi-region liquid biopsy sampling device further comprises balance weights 25. The balance weights 25 can be metal blocks such as iron block and also have anti-corrosion coatings such as parylene coating or Parylene-C coating membrane. The balance weights 25 can be disposed in the body 11 of the enclosure 10 as shown in the figure, or disposed in the two ends of the enclosure 10.

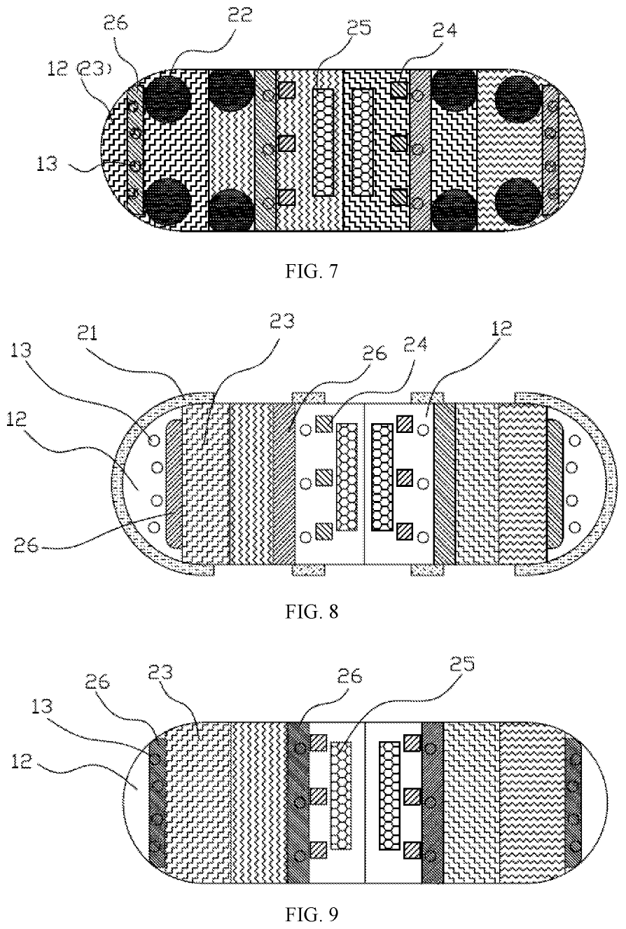

FIG. 5 shows a structural view of the digestive tract multi-region liquid biopsy sampling device according to the second embodiment of the invention. FIG. 6 shows a structural view of a piston ring as shown in FIG. 5. FIG. 7 shows a structural view of the digestive tract multi-region liquid biopsy sampling device of FIG. 5 after sampling is completed. Referring to FIGS. 5˜7, the digestive tract multi-region liquid biopsy sampling device according to the second embodiment of the invention is basically the same as the first embodiment. The difference is that in the embodiment, the sampling chamber 12 comprises a piston ring 26, and the piston ring 26 comprises a flow passage 261 for liquid to flow through the piston ring 26, wherein the piston ring 26 is configured with its outer wall against the inner wall of the enclosure 10, the expandable materials 22 are disposed at a side of the piston ring 26 away from the through holes 13, and after absorbing liquid, the expandable materials 22 expand to push the piston ring 26 to block the through holes 13.

FIG. 8 shows a structural view of the digestive tract multi-region liquid biopsy sampling device according to the third embodiment of the invention. FIG. 9 shows a structural view of the digestive tract multi-region liquid biopsy sampling device of FIG. 8 after sampling is completed. Referring to FIGS. 8˜9, the digestive tract multi-region liquid biopsy sampling device according to the third embodiment of the invention is basically the same as the second embodiment. The difference is that in the embodiment, granular expandable materials 22 are not arranged, and the water absorbing materials 23 can act as the expandable materials 22. The water absorbing materials 23 are disposed at a side of the piston rings 26 away from the through holes 13. After absorbing liquid, the water absorbing materials 23 expand to push the piston rings 26 to block the through holes 13. Preferably, in the embodiment, before absorbing liquid, the water absorbing materials 23 can be a compressed water absorbing sponge. That is, in the embodiment, the water absorbing materials 23 can maintain liquid and push the piston rings 26.

In summary, the present invention provides an enclosure 10 and a plurality of sampling chambers 12 in the enclosure 10 with through holes 13 cut in the wall of the enclosure 10 corresponding to each sampling chamber 12. Different targeted dissolution membranes 21 that can dissolve at different targeted regions cover the through holes 13 of each sampling chamber 12. In addition, expandable materials 22 that can expand after absorbing liquid are arranged in the enclosure 10. Therefore, the liquids at a plurality of regions can be accurately sampled without external power source and the through holes 13 can be blocked by the expandable materials 22 to protect the sampled liquids from being contaminated by the liquid at other regions.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in particular the matters of shape, size and arrangement of parts within the principles of the present disclosure, up to and including the full extent established by the broad general meaning of the terms used in the claims.

What is claimed is:

1. A digestive tract multi-region liquid biopsy sampling device, comprising:
an enclosure, wherein the enclosure comprises a plurality of sampling chambers formed in the enclosure, through holes cut in the wall of the enclosure corresponding to each sampling chamber, and targeted dissolution membranes covering the through holes on different sampling chambers that dissolve at different targeted regions; and
expandable materials that absorb liquids, arranged in the sampling chambers at the positions of the expandable materials corresponding to the through holes, wherein the expandable materials expand after absorbing liquid and close the through holes.

2. The digestive tract multi-region liquid biopsy sampling device of claim 1, wherein the enclosure is in a shape of capsule.

3. The digestive tract multi-region liquid biopsy sampling device of claim 1, wherein each sampling chamber comprises water absorbing materials that absorb liquid.

4. The digestive tract multi-region liquid biopsy sampling device of claim 3, wherein the expandable materials are a polymer compounded by rubber-based polymer and water-absorbent resin, and the expandable materials expand after absorbing liquid to block the through holes, so that the through holes are closed.

5. The digestive tract multi-region liquid biopsy sampling device of claim 3, wherein the expandable materials are a polymer compounded by rubber-based polymer and water-absorbent resin,
wherein the enclosure further comprises piston rings arranged in the sampling chambers, and each piston ring comprises a flow passage for liquid to flow through the piston ring,
wherein each piston ring is configured with its outer wall against the inner wall of the enclosure, the expandable materials are disposed at a side of the piston ring away from the through holes, and after absorbing liquid, the expandable materials expand to push the piston ring to block the through holes.

6. The digestive tract multi-region liquid biopsy sampling device of claim 4, wherein the expandable materials compounded by rubber-based polymer and water-absorbent resin are granular, and the granular expandable materials are disposed on the water absorbing materials or the inner wall of the enclosure.

7. The digestive tract multi-region liquid biopsy sampling device of claim 5, wherein the expandable materials compounded by rubber-based polymer and water-absorbent resin are granular, and the granular expandable materials are disposed on the water absorbing materials or the inner wall of the enclosure.

8. The digestive tract multi-region liquid biopsy sampling device of claim 1, wherein the expandable materials are water absorbing materials, the enclosure further comprises piston rings, and each piston ring comprises a flow passage for liquid to flow through the piston ring,
wherein each piston ring is configured with its outer wall against the inner wall of the enclosure, the water absorbing materials are disposed at a side of the piston ring away from the through holes, and after absorbing liquid, the water absorbing materials expand to push the piston ring to block the through holes.

9. The digestive tract multi-region liquid biopsy sampling device of claim 8, wherein before absorbing liquid, the water absorbing material is in a compressed state.

10. The digestive tract multi-region liquid biopsy sampling device of claim 1, wherein the enclosure further comprises tracer particles are arranged in the enclosure to display the position of the digestive tract multi-region liquid biopsy sampling device in the digestive tract through medical imaging technique.

11. The digestive tract multi-region liquid biopsy sampling device of claim 1, wherein the enclosure further comprises balance weights disposed inside.

12. The digestive tract multi-region liquid biopsy sampling device of claim 1, wherein the targeted dissolution membrane is selected from EUDRAGIT E100, a mixture of EUDRAGIT L100-55 and EUDRAGIT Plastoid B, pectin, guar gum, or a mixture of EUDRAGIT S100 and EUDRAGIT Plastoid B.
